(19) United States
(12) Patent Application Publication
(10) Pub. No.: US 2011/0178147 A1
(43) Pub. Date: Jul. 21, 2011

(54) COMPOSITIONS AND METHODS FOR CONTROLLING PUPIL DILATION

(75) Inventors: Sukhon Likitlersuang, Irvine, CA (US); Ajay P. Parashar, Irvine, CA (US); Chetan Pujara, Irvine, CA (US)

(73) Assignee: ALLERGAN, INC., Irvine, CA (US)

(21) Appl. No.: 12/907,719

(22) Filed: Oct. 19, 2010

Related U.S. Application Data
(60) Provisional application No. 61/253,175, filed on Oct. 20, 2009.
(60) Provisional application No. 61/321,669, filed on Apr. 7, 2010.

(51) Int. Cl. A61K 31/417; A61P 27/02

(52) U.S. Cl. 514/401

(57) ABSTRACT

The disclosure provides a composition for controlling pupil dilation comprising about 0.001% to about 1.0% by weight of an alpha-adrenergic receptor antagonist selected from phentolamine, phentolamine mesylate, or a phentolamine salt; and at least one excipient selected from the group consisting of a buffer, tonicity agent, preservative, antioxidant, surfactant, solubilizer, cosolvent, and a combination thereof. A method of controlling pupil dilation in a subject in need thereof, comprising administering a therapeutically effective amount of the composition is also provided.

### RELATED APPLICATION

This application claims the benefit of U.S. Provisional Application Ser. No. 61/253,175, filed Oct. 20, 2009 and U.S. Provisional Application Ser. No. 61/321,669, filed Apr. 7, 2010, each of which is incorporated herein by reference in its entirety.

### BACKGROUND

Pupil size can vary in diameter in darkness from about 3 mm to about 9 mm. In low ambient light, those with larger pupils can suffer from light scatter, glare, halo, and related improper focus of light rays. This aberration of focus can make functioning in low ambient conditions difficult.

### BRIEF SUMMARY OF THE INVENTION

The disclosure provides compositions for controlling pupil dilation comprising about 0.001% to about 2.0% by w/v of an alpha-adrenergic receptor antagonist selected from phentolamine, phentolamine mesylate, and a phentolamine salt; about 0.02% to about 4% w/v of a buffer, about 0.01% to about 0.75% w/v of a tonicity agent, 0.08% to about 5% w/v of an antioxidant, about 0.005% to about 2.5% w/v of a surfactant, about 0.001% to about 3% w/v of a solubilizer, and about 1% to about 25% w/v of a cosolvent by weight, and a pH of about 5.5 or between 2 and 7.

The disclosure further provides methods of controlling pupil dilation in a subject in need thereof, comprising administering a therapeutically effective amount of a composition comprising about 0.001% to 2.0% w/v, 0.01% about 1.0% by w/v, 0.02-1.0% w/v, 0.02-1.0% w/v, 0.03-1.0% w/v, 0.04-1.0% w/v, 0.05-1.0% w/v, 0.06-1.0% w/v, 0.07-1.0% w/v, 0.08-1.0% w/v, 0.09-1.0% w/v, 0.1-1.0% w/v, 0.2-1.0% w/v, 0.3-1.0% w/v, 0.4-1.0% w/v, 0.5-1.0% w/v, 0.6-1.0% w/v, 0.7-1.0% w/v, 0.8-1.0% w/v, 0.9-1.0% w/v, 1.1% w/v, 1.2% w/v, 1.3% w/v, 1.4% w/v, 1.5% w/v, 1.6% w/v, 1.7% w/v, 1.8% w/v, 1.9% w/v or 2.0% w/v of an alpha-adrenergic receptor antagonist selected from phentolamine, phentolamine mesylate, and a phentolamine salt; about 0.02% to about 4% of a buffer, about 0.01% to about 0.75% of a tonicity agent, 0.08% to about 5% of an antioxidant, about 0.005% to about 2.5% of a surfactant, about 0.001% to about 3% of a solubilizer, and about 1% to about 25% of a cosolvent by w/v, and a pH of about 5.5. U.S. Pat. No. 7,229,630 is hereby incorporated by reference.

### DETAILED DESCRIPTION

The disclosure provides compositions and methods for controlling pupil dilation in a subject in need thereof. The compositions comprise about 0.001% to 2.0% by w/v or 0.01% to about 1.0% by w/v of an alpha-adrenergic receptor antagonist selected from phentolamine, phentolamine mesylate, or another phentolamine salt; and at least one excipient selected from the group consisting of a buffer, tonicity agent, preservative, antioxidant, surfactant, solubilizer, cosolvent, and a combination thereof, and a pH of about 5.5.

The disclosure further provides a method of controlling pupil dilation in a subject in need thereof, comprising administering a therapeutically effective amount of one of the disclosed compositions. The amount and frequency of administration can vary according to the need and underlying condition of the subject. The composition can be administered prior to, during, or after operative procedures. Further, the composition can be administered on a daily (i.e., 1, 2, 3, 4, or more times per day), bi- or tri-weekly, weekly, or on a monthly basis, or on an as-needed basis (for example in low ambient light situations), and can be administered for a finite period of time or indefinitely in order to treat a persistent or transient need.

As used herein, an alpha-1 antagonist binds to the alpha-1 adrenergic receptor. Preferably, the alpha-1 adrenergic receptor is a selective dilator of the iris smooth muscle. The alpha-1 antagonist can be in the phentolamine family, known as imidazolines, an alkylating agent, such as phenoxybenzamine, or a piperazinyl quinazoline with more potent alpha-1 adrenergic antagonist activity than dapiprazole. In certain embodiments, phentolamine, phentolamine mesylate, and/or a phentolamine salt is used. Alpha-1 adrenergic antagonists, such as phentolamine, phentolamine mesylate, and phentolamine salts, inhibit pupil dilation and are therefore effective in controlling pupil dilation. Phentolamine is a water soluble drug molecule that can be topically delivered to the eye for treatment of a condition in which pupil dilation is abnormal. Although phentolamine is referred to throughout this disclosure, it is intended to include phentolamine mesylate, and/or phentolamine salt as well.

The compositions comprise or consist of a therapeutically effective amount of the alpha-1 antagonist. In this regard, the alpha-1 antagonist which can be phentolamine, phentolamine mesylate, and a phentolamine salt can be present in a composition in an amount of about 0.001% to about 2.0% by weight, or about 0.01%-0.9% w/v, or 0.01% to about 0.8% by w/v, 0.01% to about 0.7% w/v, or 0.01% to about 0.6% w/v, 0.01% to about 0.5% w/v, or about 0.01% to about 0.4% by w/v, or 0.01% to about 0.3% by w/v, 0.01% to about 0.1% by w/v, 0.01%, 0.02%, 0.03%, 0.04%, 0.05%, 0.06%, 0.07%, 0.08%, and 0.09% w/v. The concentration of the alpha-1 antagonist in the composition depends on various factors, such as composition form (i.e., solution, emulsion, ointment, etc), route of administration, age and health status of the subject, the underlying condition, and desired outcome.

While the composition can be used to optimize/control pupil size under any circumstance, the composition is administered to the eye of a subject to reduce naturally occurring pupil dilation in low ambient light, particularly in situations where the dilation is excessive such that it affects visual acuity. The composition can be used also to counteract pupil dilatation caused by medication.

The phrase “low ambient light” as used herein, refers to a light environment in which the pupils of the subject are dilated to a substantially maximum amount. Alternatively, the term “bright light” as used herein describes a surrounding light environment wherein the pupil of the subject's eye is contracted maximally, that is, dilated to a minimum amount.

The alpha-1 antagonist utilized in the disclosed composition limits pupil dilation but does not significantly affect pupillary constriction. Therefore, the composition is particularly useful in subjects with large pupils in low ambient light, whose low ambient light pupil diameter exceeds their daylight pupil diameter considerably. In contrast, the composition can have less effect on pupil diameter in patients who have a more idealized pupil diameter in low ambient light and exhibit a low ambient light pupil that is nearly equal to their daylight pupil.

The composition can be administered directly (i.e., via drops) or indirectly to an eye. In this regard, the composition can be administered indirectly by placing the composition on an article, such as a contact lens, and then placing the article onto the eye. The dosage and frequency of administration will vary according to the needs of the individual subject.

Typically a 3 mm pupil diameter is sufficiently large to allow light to enter the eye in low ambient light situations, yet provide enough filter to minimize light scatter of ambient artificial light and or point sources of light. In contrast, a 9 mm pupil diameter utilizes nine times more corneal surface area, and induces considerable light scatter of point sources of light. The variability of pupil size in low ambient light and refractive optics that add to light scatter (such as glasses, contact lenses) can create a circumstance in which a subject has difficulty navigating in low ambient light situations as a result of glare, halo, and related distortions. Accordingly, it is desirable to maintain an optimal pupil diameter, such as about 2.5 mm to about 6 mm, preferably about 3 mm to about 5 mm. Accordingly, pupil dilation is considered to be “controlled” when a desirable pupil diameter is achieved or maintained in low ambient light situations, as described herein. Pupil dilation control can include a reduction in the size of a pupil, or the maintenance of a desired level of pupil dilation.

The subject in need of pupil dilation control can be a mammal of any gender or age. Preferably, the subject is a human. In certain instances, the subject has undergone a surgical procedure that caused an increase the degree of light scatter in low ambient lighting, such as LASIK™, or placement of a corneal prosthesis, such as an intraocular lens. The disclosed composition can maintain the pupil size at about 3 mm to about 5 mm.

The disclosed composition provides improved quality of vision in low ambient light without negative clinical effects in normal lighting conditions. The composition can be used to optimize pupil size to obtain enhanced vision acuity in low ambient light by reducing the pupil diameter in low ambient light without substantially reducing the size of the pupil in bright light.

In certain embodiments, the optimized pupil diameter in low ambient light is no more than about 200% greater than that in bright light. In other embodiments, the pupil diameter in low ambient light is no more than about 150%, about 100%, about 75%, about 60%, about 50%, or no more than about 33% greater than that in bright light.

The composition can be in suitable form for topical administration. In certain embodiments, the composition is a solution, a suspension, an emulsion, an ointment, a gel, or a solid insert. The disclosure includes microemulsions and reverse emulsions (i.e., water in oil). Microemulsions are clear, stable, isotropic liquid mixtures of oil, water and a surfactant, frequently in combination with a cosurfactant. Non-limiting examples of compositions in accordance with certain embodiments of the invention are disclosed in Table 1 and Table 2 (preservative-free formulations).

In certain embodiments, the alpha-1 antagonist is a phentolamine. Phentolamine lacks chemical stability in aqueous media. Therefore, excipients that can enhance the stability of phentolamine, as well as other alpha-1 antagonists, can be utilized to formulate the agent at dose strengths sufficient for improving visual acuity.

The composition comprises at least one excipient. Excipients and additives suitable for use in the disclosed composition are known to those of skill in the art and include without limitation, carriers, stabilizers, solubilizers, tonicity enhancing agents, buffers, preservatives, thickeners, complexing agents, and combinations thereof.

Carriers used in the disclosed composition are suitable for topical administration, and include, for example, water, mixtures of water and water-miscible solvents, such as C₁-C₇ alkanols, vegetable oils or mineral oils comprising from about 0.1 to about 30%, 0.5 to about 15%, or about 0.5 to about 5% or 0.1-1.0% w/v, 0.2-1.0% w/v, 0.3-1.0% w/v, 0.4-1.0% w/v, 0.5-1.0% w/v, 0.6-1.0% w/v, 0.7-1.0% w/v, 0.8-1.0% w/v, 0.9-1.0% w/v, 1.1% w/v, 1.2% w/v, 1.3% w/v, 1.4% w/v, 1.5% w/v, 1.6% w/v, 1.7% w/v, 1.8% w/v, 1.9% w/v or 2.0% w/v hydroxyethylcellulose, ethyl oleate, carboxymethylcellulose, polyvinylpyrrolidone and other non-toxic water-soluble polymers suitable for ophthalmic use including cellulose derivatives such as methylcellulose; alkali metal salts of carboxymethylcellulose, hydroxymethylcellulose, hydroxyethylcellulose, methylhydroxypropylcellulose and hydroxypropylcellulose, acrylates; or methacrylates such as salts of polyacrylic acid or ethyl acrylate, polyacrylamides; natural products, such as gelatin, alginates, pectins, tragacanth, karaya gum, xanthan gum, carrageenin, agar and acacia; starch-derivatives such as starch acetate and hydroxypropyl starch; synthetic products such as polyvinyl alcohol, polyvinylpyrrolidone, polyvinyl methyl ether, polyethylene oxide; cross-linked polyacrylic acid such as neutral CARBOPOL®, and/or combinations thereof. The concentration of the carrier is, for example, from 1 to 100,000 times the concentration of the active ingredient. In certain embodiments, the carrier is water.

In certain embodiments, the composition comprises a cosolvent. The cosolvent can be selected from, but not limited to, glycerin and mannitol, hyalauronic acid and others. The cosolvent can be present in the composition in an amount of about 1% to about 25% by weight, about 3% to about 15% by w/v, or from about 4 to about 12% by w/v, or from about 5 to about 10% by w/v.

Solubilizers suitable for use in the composition include, but are not limited to, tyloxapol, fatty acid glycerol poly-lower alkylene (i.e., C₁ to C₇, linear or branched) glycol esters, fatty acid poly-lower alkylene glycol esters, polyethylene glycols, glycerol ethers vitamin E and vitamin E derivatives, such as vitamin E tocopherol polyethylene glycol 1000 succinate (TPGS) or mixtures of those compounds. The concentration used depends on the formulation of the composition and is typically sufficient to solubilize the active ingredient. Typically, the solubilizer is present in an amount of about 0.1 to about 5000 times the concentration of the active ingredient, i.e., the alpha-1 antagonist. In certain embodiments, the solubilizer is present in an amount of about 0.001% to about 3% by w/v, or about 0.009% to about 2% by w/v, or about 0.01% to about 2.0% by w/v, 0.02-1.0% w/v, 0.02-1.0% w/v, 0.03-1.0% w/v, 0.04-1.0% w/v, 0.05-1.0% w/v, 0.06-1.0% w/v, 0.07-1.0% w/v, 0.08-1.0% w/v, 0.09-1.0% w/v, 0.1-1.0% w/v, 0.2-1.0% w/v, 0.3-1.0% w/v, 0.4-1.0% w/v, 0.5-1.0% w/v, 0.6-1.0% w/v, 0.7-1.0% w/v, 0.8-1.0% w/v, 0.9-1.0% w/v, 1.1% w/v, 1.2% w/v, 1.3% w/v, 1.4% w/v, 1.5% w/v, 1.6% w/v, 1.7% w/v, 1.8% w/v, 1.9% w/v or 2.0% w/v.

The composition can comprise a surfactant/emulsifier. Surfactants suitable for use in the disclosed composition include, but are not limited to, Polysorbate 20, Polysorbate 40, Polysorbate 60, and Polysorbate 80, glyceryl stearate, isopropyl stearate, polyoxyl stearate, propylene glycol stearate, and sucrose stearate, polyethylene glycol, polyethylene oxides, polypropylene oxides, polyethylene oxide, polypropylene oxide copolymers, alcohol ethoxylates, and alkylphenol ethoxylates, alkyl glycoside, alkyl polyglycoside, fatty alcohol, hydroxypropylmethyl cellulose (HPMC) and carboxymethyl cellulose (CMC), polyacrylic acid, including, but not limited to, a Carbomer, phosphatidyl chloline and phosphatidyl serine, as well as those listed in U.S. Pat. No. 7,276,476, hereby incorporated by reference, Typically, a surfactant is present in an amount of about 0.005% to about 2.5% by w/v or about 0.01% to about 2.0 by w/v or about 0.01% to about 1.0% by w/v, 0.02-1.0% w/v, 0.02-1.0% w/v, 0.03-1.0% w/v, 0.04-1.0% w/v, 0.05-1.0% w/v, 0.06-1.0% w/v, 0.07-1.0% w/v, 0.08-1.0% w/v, 0.09-1.0% w/v. The emulsions of the disclosed compositions can be stabilized using one or more polyelectrolytes from the family of cross-linked polyacrylates, such as carbomers and PEMULEN® (Hoffman La-Roche). Pemulens are high molecular w/v co-polymers of acrylic acid and a long chain alkyl methacrylate cross-linked with allyl ethers of pentaerythritol. They contain not less than about 52% and not more than about 62% of carboxylic acid groups. The viscosity of a neutralized 1.0% aqueous dispersion is between about 9,500 and about 26,500 centipoise. Exemplary emulsion formulations are shown in Table 3.

The pH of the composition is about 4 to about 6.5, or about 5.0 to about 6. In certain embodiments, the pH is about 5.5. Accordingly, the composition can comprise a buffer selected from the group consisting of acetate, ascorbate, borate, hydrogen carbonate, carbonate, citrate, gluconate, lactate, phosphate, propionate, perborate, tris-(hydroxymethyl)amineomethane (TRIS), bis(2-hydroxyethyl)-imino-tris-(hydroxymethyl)aminoalcahol(bis-tris), N-[2-hydroxy-1,1-bis(hydroxymethyl)ethyl]glycine (tricene), N-[2-hydroxy-1,1-bis(hydroxymethyl)ethyl]glycine, MOPS, N-(carbamoylmethyl)taurine (ACES), amino acid, amino acid derivatives, and a combination thereof. The amount of buffer substance added is that necessary to ensure and maintain a physiologically tolerable pH range. Typically, the buffer is present in an amount of about 0.02% to about 4.0% by w/v. In certain embodiments, the buffer is present in an amount of about 0.05% to about 3.0% by w/v or about 0.05% to about 2.5% by w/v.

The composition can comprise a tonicity enhancing agent to approximate the osmotic pressure of normal lacrimal fluid, which is equivalent to a 0.9% solution of sodium. Suitable tonicity enhancing agents may include, for example, ionic compounds such as alkali metal or alkaline earth metal halides such as CaCl, KBr, KCl, LiCl, NaI, NaBr, NaCl, MgCl, or boric acid. Non-ionic tonicity enhancing agents include, for example, urea, glycerol, sorbitol, mannitol, propylene glycol, or dextrose. In certain embodiments, the tonicity enhancing agent is NaCl, KCl, dextrose, CaCl, MgCl, dextrose, and a combination thereof. The tonicity enhancing agent can be present in an amount of about 0.01% to about 0.75%, or about 0.1% to about 0.5% by w/v or 0.01% about 1.0% by w/v, 0.02-1.0% w/v, 0.02-1.0% w/v, 0.03-1.0% w/v, 0.04-1.0% w/v, 0.05-1.0% w/v, 0.06-1.0% w/v, 0.07-1.0% w/v, 0.08-1.0% w/v, 0.09-1.0% w/v, 0.1-1.0% w/v. The osmolality of the composition can be about 50 to about 1000 mOsm/kg, or about 100 to about 400 mOsm/kg. In certain embodiments, the osmolality can be about 200 to about 400 mOsm/kg or about 280 to about 380 mOsm/kg.

The disclosed composition can optionally include a preservative or no preservatice in unit dose form. A preservative is particularly desirable for use with multi-dose packaging configurations. Examples of suitable preservatives include, but are not limited to, quaternary ammonium salts such as cetrimide, benzalkonium chloride or benzoxonium chloride; alkyl-mercury salts of thiosalicylic acid such as thiomersal, phenylmercuric nitrate, phenylmercuric acetate or phenylmercuric borate; parabens such as methylparaben or propylparaben; alcohols such as chlorobutanol, benzyl alcohol or phenyl ethanol; chlorine dioxide or PURITE, guanidine derivatives such as chlorohexidine, or sorbic acid. In certain embodiments, the preservative is benzalkonium chloride. Where appropriate, a sufficient amount of preservative is added to the ophthalmic composition to ensure protection against secondary contamination during use caused by bacteria and fungi. In certain embodiments, the preservative is present in an amount of about 0.001% to about 1% by w/v, or about 0.02 to about 0.5% by w/v or 0.01% about 1.0% by w/v, 0.02-1.0% w/v, 0.02-1.0% w/v, 0.03-1.0% w/v, 0.04-1.0% w/v, 0.05-1.0% w/v, 0.06-1.0% w/v, 0.07-1.0% w/v, 0.08-1.0% w/v, 0.09-1.0% w/v, 0.1-1.0% w/v.

To increase the stability of the formulation and decrease alpha-1 antagonist degradation or to slow the degradation rate, the composition can further comprise antioxidants, such as, ascorbic acid, vitamin E, N-acetylcarnosine (NAC), sorbic acid, ethylene diamine tetraacetic acid (EDTA), and a combination thereof. The antioxidant can be present in the composition in an amount of about 0.001% to about 30% by w/v. In certain embodiments, the antioxidant is present in an amount of about 0.01% to about 15% by w/v or about 0.8% to about 5% by w/v.

The composition disclosed herein can further comprise one or more non-toxic excipients, such as, for example, wetting agents, fillers, and polyethylene glycols. Additional excipients can be used, such as acetylcysteine, cysteine, sodium hydrogen sulfite, butyl-hydroxyanisole, butyl-hydroxytoluene or alpha-tocopherol acetate; cyclodextrin, thiourea, thiosorbitol, sodium dioctyl sulfosuccinate or monothioglycerol, lauric acid sorbitol ester, triethanol amine oleate or palmitic acid ester. The amount and type of excipient added is in accordance with the particular requirements of the composition and is generally in the range of from about 0.0001% to about 90% by w/v.

The composition can be packaged as a single or multi-dose dropper bottle or in a unit dose vial. Preferably, when a multi-dose packaging configuration is used, the composition comprises a preservative such as benzalkonium chloride.

In certain embodiments, the composition can further comprise an additional active agent, including but not limited to, an antibiotic, an anti-allergic, a local anesthetic, an additional ophthalmic agent, and combinations thereof.

Certain embodiments of the disclosed compositions may optionally incorporate a local anesthetic, which can be selected from the group of ambucaine, amolanone, amylocalne, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethysoquin, dimethocaine, diperodon, dycyclonine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine, phenol, piperocaine, piridocaine, polidocanol, pramoxine, prilocalne, procaine, propanocaine, proparacaine, propipocaine, propoxycaine, psuedococaine, pyrrocaine, ropivacaine, salicyl alcohol, tetracaine, tolycaine, trimecaine, zolamine, and salts thereof. The concentration of the local anesthetic in the compositions described herein can be therapeutically effective, meaning the concentration is adequate to provide a therapeutic benefit without inflicting harm to the patient.

The compositions may optionally comprise an opthalmologically acceptable anti-inflammatory agent, such as any non-steroidal anti-inflammatory drug (NSAID) in an amount effective to reduce inflammation in an eye. Non-limiting examples include agents that inhibit the cycloxygenase (COX)-1 and/or -2 enzyme, including but not limited to propionic acids such as naproxen, flurbiprofen, oxaprozin, ibuprofen, ketoprofen, fenoprofen; ketorolac tromethamine; acetic acid derivatives such as sulindac, indomethacin, and etodolac; phenylacetic acids such as diclofenac, bromfenac, and suprofen; arylacetic prodrugs such as nepafenac, and amfenac; salicyclic acids, such as aspirin, salsalate, diflunisal, choline magnesium trisalicylate (CMT); para-aminophenol derivatives such as acetaminophen; naphthylalkanones such as nabumetone; enolic acid derivatives such as piroxicam and meloxicam; femanates such as mefenamic acid, meclofenamate and flufenamic acid; pyrroleacetic acids such as tolmetin; and pyrazolones such as phenylbutazone; COX-2 selective inhibitors such as celecoxib, valdecoxib, parecoxib, etoricoxib, and luaricoxib; including all esters and pharmaceutically acceptable salts thereof. A steroidal anti-inflammatory agent can also be incorporated, in certain embodiments, and can include, without limitation, hydrocortisone, cortisone, prednisolone, and prednisone.

Antimicrobial agents suitable for use in the disclosed compositions include, but are not limited to, antibiotics such as aminoglycosides such as gentamycin, kanamycin, neomycin, and vancomycin; amphenicols such as chloramphenicol; cephalosporins, such as cefazolin HCl; penicillins such as ampicillin, penicillin, carbenicillin, oxycillin, methicillin; lincosamides such as lincomycin; polypeptide antibiotics such as polymixin and bacitracin; tetracyclines such as tetracycline; quinolones such as ciproflaxin, etc.; sulfonamides such as chloramine T; and sulfones such as sulfanilic acid as the hydrophilic entity; as well as anti-viral drugs, e.g. acyclovir, gancyclovir, vidarabine, azidothymidine, dideoxyinosine, and dideoxycytosine. Antifungal agents and any other opthalmically suitable antimicrobials are contemplated herein as well.

TABLE 1

| Ingredient | Unit | Formulation 1 | Formulation 2 |
|---|---|---|---|
| Phentolamine, | % w/v | 0.01-0.5, 0.02, | 0.01-0.5, 0.02, 0.03, |
| Phentolamine | | 0.03, 0.04, 0.05, | 0.04, 0.05, 0.06, |
| Mesylate, or | | 0.06, 0.07, | 0.07, 0.080.09, .1, .2, |
| Phentolamine | | 0.080.09, | .3, .4, .5, .6, .7, .8, |
| salt | | .1, .2, .3, .4, .5 | .9. 1.0 |
| Tris Amino | % w/v | 0.1-0.5 or 0.268 | 0.1-1.0 or 0.268 |
| EDTA | % w/v | 0.01-0.1 or 0.014 | 0.1-1.0 or 0.014 |
| NaCl | % w/v | 0.1 | — |
| Mannitol | % w/v | — | 5 |
| Glycerin | % w/v | 10 | — |
| Benzalkonium | % w/v | 0.02 | 0.02 |
| Chloride | | | |
| Ascorbic Acid | % w/v | 0.1 | 0.1 |
| NaOH/HCl | pH | 505 | 5.5 |
| Purified | Q.S. | Q.S. | Q.S. |
| Water/WFI | | | |

TABLE 2

| Ingredient | Unit | Formulation 3 | Formulation 4 |
|---|---|---|---|
| Phentolamine, | % w/v | 0.01-0.5 | 0.01-0.5 |
| Phentolamine | | | |
| Mesylate, or | | | |
| Phentolamine salt | | | |
| Tris Amino | % w/v | 0.268 | 0.268 |
| EDTA | % w/v | 0.014 | 0.014 |
| NaCl | % w/v | 0.1 | — |
| Mannitol | % w/v | — | 5 |
| Glycerin | % w/v | 10 | — |
| Ascorbic Acid | % w/v | 0.1 | 0.1 |
| NaOH/HCl | pH | 5.5 | 5.5 |
| Purified Water/WFI | Q.S. | Q.S. | Q.S. |

TABLE 3

| | | Formu- | Formu- | Formu- | Formu- |
|---|---|---|---|---|---|
| | | lation | lation | lation | lation |
| Ingredient | Unit | 5 | 6 | 7 | 8 |
| Phentolamine, | % w/v | 0.1 | 0.2 | 0.3 | 0.4 |
| Phentolamine | | | | | |
| Mesylate, or | | | | | |
| Phentolamine | | | | | |
| salt | | | | | |
| Tris Amino | % w/v | 0.268 | 0.268 | 0.268 | 0.268 |
| EDTA | % w/v | 0.014 | 0.014 | 0.014 | 0.014 |
| NaCl | % w/v | 0.1 | — | 0.1 | 0.1 |
| Mannitol | % w/v | — | 5 | 5 | |
| Glycerin | % w/v | 10 | — | — | 10 |
| Benzalkonium | % w/v | 0.02 | 0.02 | — | — |
| Chloride | | | | | |
| Polysorbate | % w/v | 0.01 | — | 1.0 | |
| 20 | | | | | |
| Polysorbate | | — | — | — | 1.0 |
| 40 | | | | | |
| Carboxy | % w/v | — | 0.5 | — | — |
| methyl | | | | | |
| cellulose | | | | | |
| Ascorbic Acid | % w/v | 0.1 | 0.1 | 0.1 | 0.1 |
| NaOH/HCl | pH | 5.5 | 5.5 | 5.5 | 5.5 |
| Purified | Q.S. | Q.S. | Q.S. | Q.S. | Q.S. |
| Water/WFI | | | | | |

A range of different alpha-1 antagonists are known to those skilled in the art. The present disclosure includes those compounds and equivalent compounds which have substantially the same therapeutic effect as the present invention.

### Example 1

### Treatment Example

A 29 year old woman presents with difficulty driving at night following LASIK™ surgery. The patient is diagnosed as having enlarged pupil diameter and is treated with the composition of Formula 1 daily for 1 week. The patient experiences improved vision in low ambient light situations and does not have difficulty driving at night following treatment.

### Example 2

### Alternate Treatment Example

A 50 year old man presents with difficulty seeing in low ambient light conditions due to abnormally dilated pupils or a stiffened cornea. The patient is treated with the composition of Formula 2. The patient experiences a reduction in pupil diameter in low ambient light conditions and improved vision.

### Example 3

### Alternate Treatment Example

A 29 year old man experiences halo and distorted vision in dimly lit environments. He is diagnosed as having abnormally large pupil diameter in low ambient light and is treated with the composition of Formula 3. Following treatment, the patient's vision is improved in low ambient light and he no longer experiences halo or distortion.

### Example 4

### Alternate Treatment Example

A 62 year old woman experiences difficulty focusing in low ambient light after receiving an intraocular lens in her left eye. She is diagnosed as having an abnormally large pupil diameter in the eye and is treated with the composition of Formula 5. Following treatment, her vision is improved and she is able to focus in low ambient light conditions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). embodiments of the invention so claimed are inherently or expressly described and enabled herein.

What is claimed:

1. A composition for controlling pupil dilation comprising about 0.001% to about 1.0% by w/v of an alpha-adrenergic receptor antagonist selected from the group consisting of phentolamine, phentolamine mesylate, or a phentolamine salt; about 0.02% to about 4% of a buffer, about 0.01% to about 0.75% of a tonicity agent, 0.08% to about 5% of an antioxidant, about 0.005% to about 2.5% of a surfactant, about 0.001% to about 3% of a solubilizer, and about 1% to about 25% of a cosolvent by weight, and a pH of about 5.5.

2. The composition of claim 1, further comprising about 0.001 to about 1% of a preservative.

3. The composition of claim 1, wherein the buffer is selected from the group consisting of carbonate, tris-(hydroxymethyl)amineomethane (TRIS), bis(2-hydroxyethyl)-imino-tris-(hydroxymethyl)aminoalcahol (bis-tris), N-[2-hydroxy-1,1-bis(hydroxymethyl)ethyl]glycine (tricene), N-[2-hydroxy-1,1-bis(hydroxymethyl)ethyl]glycine, MOPS, N-(carbamoylmethyl)taurine (ACES), amino acid, amino acid derivatives, and a combination thereof.

4. The composition of claim 1, wherein the tonicity agent is selected from the group consisting of sodium chloride, potassium chloride, dextrose, calcium chloride, magnesium chloride, and a combination thereof.

5. The composition of claim 1, wherein the alpha-adrenergic is phentolamine mesylate.

6. The composition of claim 1, wherein the antioxidant is selected from the group consisting of ascorbic acid, vitamin E, N-acetylcarnosine (NAC), sorbic acid, EDTA, and a combination thereof.

7. The composition of claim 1, wherein the composition comprises about 0.268% Tris Amino, 0.14% EDTA, 0.1% NaCl, 10% glycerin, 0.02% benzalkonium chloride, 0.1% ascorbic acid, and a pH of about 5.5.

8. The composition of claim 1, wherein the composition comprises about 0.268% Tris Amino, 0.14% EDTA, 5% mannitol, 0.02% benzalkonium chloride, 0.1% ascorbic acid, and a pH of about 5.5.

9. The composition of claim 1, wherein the composition comprises about 0.268% Tris Amino, 0.14% EDTA, 0.1% NaCl, 10% glycerin, 0.1% ascorbic acid, and a pH of about 5.5.

10. The composition of claim 1, wherein the composition comprises about 0.268% Tris Amino, 0.14% EDTA, 5% mannitol, 0.1% ascorbic acid, and a pH of about 5.5.

11. The composition of claim 1, wherein the composition is a solution.

12. The composition of claim 11, wherein the emulsion is selected from a group consisting of a reverse emulsion, a microemulsion, an oil-in-water emulsion, and a water-in-oil emulsion.

13. A method of controlling pupil dilation in a subject in need thereof, comprising administering a therapeutically effective amount of a composition comprising about 0.001% to about 1.0% by w/v of an alpha-adrenergic receptor antagonist selected from phentolamine, phentolamine mesylate, and a phentolamine salt; and about 0.02% to about 4% of a buffer, about 0.01% to about 0.75% of a tonicity agent, 0.08% to about 5% of an antioxidant, about 0.005% to about 2.5% of a surfactant, about 0.001% to about 3% of a solubilizer, and about 1% to about 25% of a cosolvent by w/v, wherein pupil dilation in the subject is controlled.

14. The method of claim 13, further comprising about 0.001 to about 1% of a preservative.

15. The method of claim 13, wherein the composition comprises about 0.268% Tris Amino, 0.14% EDTA, 0.1% NaCl, 10% glycerin, 0.02% benzalkonium chloride, 0.1% ascorbic acid, and a pH of about 5.5.

16. The method of claim 13, wherein the composition comprises about 0.268% Tris Amino, 0.14% EDTA, 5% mannitol, 0.02% benzalkonium chloride, 0.1% ascorbic acid, and a pH of about 5.5.

17. The method of claim 13, wherein the composition comprises about 0.268% Tris Amino, 0.14% EDTA, 0.1% NaCl, 10% glycerin, 0.1% ascorbic acid, and a pH of about 5.5.

18. The method of claim 13, wherein the composition comprises about 0.268% Tris Amino, 0.14% EDTA, 5% mannitol, 0.1% ascorbic acid, and a pH of about 5.5.

19. The method of claim 13, wherein the composition is administered daily, weekly, or monthly.

20. The method of claim 13, wherein the pupil dilation is controlled in low ambient light.
